(19) United States
(12) Patent Application Publication
(10) Pub. No.: US 2011/0259350 A1
(43) Pub. Date: Oct. 27, 2011

(54) Methods of Using SDF-1 (CXCL12) as a Diagnostic and Mesenchymal Stem Cell (Multipotent Stromal Cell)-Specific Therapeutic Biomarker for the Treatment of Kidney Injury and Other Major Organs

(75) Inventor: Christof Westenfelder, Salt Lake City, UT (US)

(73) Assignee: ALLOCURE, INC., Salt Lake City, UT (US)

(21) Appl. No.: 13/125,605

(22) PCT Filed: Oct. 22, 2009
(86) PCT No.: PCT/US2009/005776

§ 371 Date: Jul. 12, 2011

Related U.S. Application Data
(60) Provisional application No. 61/107,468, filed on Oct. 22, 2008.

(51) Int. Cl. A61B 19/00; G01N 33/53; A61P 13/12; A61K 35/12

(52) U.S. Cl. 128/898; 424/93.7; 435/7.92

(57) ABSTRACT

This application describes the detection and diagnosis of kidney pathology including acute kidney injury by the detection of changes in the amounts of biomarkers in the urine of patients. These biomarkers include stromal derived factor (SDF-1 or CXCL12).

### RELATED APPLICATIONS

This application claims priority to U.S. Provisional Application No. 61/107,468, filed on Oct. 22, 2008, the contents of which are incorporated herein by reference in their entirety.

### FIELD OF THE INVENTION

This application relates to the detection, diagnosis and treatment of kidney diseases and those of other major organs (e.g., liver, heart, brain, pancreas, lungs) including acute kidney injury by the detection of changes in the amounts of SDF-1 or CXCL12 as a biomarkers in the urine and serum of patients.

### BACKGROUND

Acute kidney injury (AKI) is defined as an acute deterioration in renal excretory function within hours or days, resulting in the accumulation of “uremic toxins,” and, importantly, a rise in the blood levels of potassium, hydrogen and other ions, all of which contribute to life threatening multisystem complications such as bleeding, seizures, cardiac arrhythmias or arrest, and possible volume overload with pulmonary congestion and poor oxygen uptake. The most common cause of AKI is an ischemic insult of the kidney resulting in injury of renal tubular and postglomerular vascular endothelial cells. The principal etiologies for this ischemic form of AKI include intravascular volume contraction, resulting from bleeding, thrombotic events, shock, sepsis, major cardiovascular surgery, arterial stenoses, and others. Nephrotoxic forms of AKI can be caused by radiocontrast agents, significant numbers of frequently used medications such as chemotherapeutic drugs, antibiotics and certain immunosuppressants such as cis-Platinum and cyclosporine. Patients most at risk for all forms of AKI include diabetics, those with underlying kidney, liver, cardiovascular disease, the elderly, recipients of a bone marrow transplant, and those with cancer or other debilitating disorders.

Both ischemic and nephrotoxic forms of AKI result in dysfunction and death of renal tubular and microvascular endothelial cells. Sublethally injured tubular cells dedifferentiate, lose their polarity and express vimentin, a mesenchymal cell marker, and Pax-2, a transcription factor that is normally only expressed in the process of mesenchymal-epithelial transdifferentiation in the embryonic kidney. Injured endothelial cells also exhibit characteristic changes.

The kidney, even after severe acute insults, has the remarkable capacity of self-regeneration and consequent re-establishment of nearly normal function. It is thought that the regeneration of injured nephron segments is the result of migration, proliferation and re-differentation of surviving tubular and endothelial cells. However, the self-regeneration capacity of the surviving tubular and vascular endothelial cells may be exceeded in severe AKI. Patients with isolated AKI from any cause, i.e., AKI that occurs without multi organ failure (MOF), continue to have a mortality rate in excess of 50%. This dismal prognosis has not improved despite intensive care support, hemodialysis, and the recent use of atrial natriuretic peptide, insulin-like growth factor-I (IGF-I), more biocompatible dialysis membranes, continuous hemodialysis, and other interventions. An urgent need exists to enhance the kidney's self-defense and autoregenerative capacity after severe injury.

Another acute form of AKI, transplant-associated acute renal failure (TA-ARF), also termed early graft dysfunction (EGD), commonly develops upon kidney transplantation, mainly in patients receiving transplants from cadaveric donors, although TA-ARF may also occur in patients receiving a living related donor kidney. Up to 50% of currently performed kidney transplants utilize cadaveric donors. Kidney recipients who develop significant TA-ARF require treatment with hemodialysis until graft function recovers. The risk of TA-ARF is increased with elderly donors and recipients, marginal graft quality, significant comorbidities and prior transplants in the recipient, and an extended period of time between harvest of the donor kidney from a cadaveric donor and its implantation into the recipient, known as “cold ischemia time.” Early graft dysfunction or TA-ARF has serious long-term consequences, including accelerated graft loss due to progressive, irreversible loss in kidney function that is initiated by TA-ARF, and an increased incidence of acute rejection episodes leading to premature loss of the kidney graft. Therefore, a great need exists to provide a treatment for early graft dysfunction due to TA-ARF or EGD.

Chronic renal failure (CRF) or Chronic Kidney Disease (CKD) is the progressive loss of nephrons and consequent loss of renal function, resulting in End Stage Renal Disease (ESRD), at which time patient survival depends on dialysis support or kidney transplantation. The progressive loss of nephrons, i.e., glomeruli, tubuli and microvasculature, appears to result from self-perpetuating fibrotic, inflammatory and sclerosing processes, most prominently manifested in the glomeruli and renal interstitium. The loss of nephrons is most commonly initiated by diabetic nephropathy, glomerulonephritides, many proteinuric disorders, hypertension, vasculitic, inflammatory and other injuries to the kidney. Currently available forms of therapy, such as the administration of angiotensin converting enzyme inhibitors, angiotensin receptor blockers, other anti-hypertensive and anti-inflammatory drugs such as steroids, cyclosporine and others, lipid lowering agents, omega-3 fatty acids, a low protein diet, optimal weight, blood pressure and blood sugar control, particularly in diabetics, can significantly slow and occasionally arrest the chronic loss of kidney function in the above conditions. The development of ESRD can be prevented in some compliant patients and delayed others. Despite these successes, the annual growth of patient numbers with ESRD, requiring chronic dialysis or transplantation, remains at 6%, representing a continuously growing medical and financial burden. There exists an urgent need for the development of new interventions for the effective treatment of CRF or CKD and thereby ESRD, to treat patients who fail to respond to conventional therapy, i.e., whose renal function continues to deteriorate. Stem cell treatment will be provided to arrest/reverse the fibrotic processes in the kidney.

It is now well recognized that currently used laboratory tests measuring the concentration of serum creatinine (SCr) and blood urea nitrogen (BUN) for the diagnosis of clinical Acute Kidney Injury (AKI) are able to identify AKI only at 24 to 48 hrs after a given kidney insult (shock, trauma, sepsis, major surgery, drugs). This delayed diagnosis of AKI translates into delayed institution of therapeutic or preventative interventions, thereby resulting in poor outcomes, characterized by high mortality rates (greater than 50%), prolonged hospital stays, transient need for dialysis, irreversible loss of kidney function (requiring chronic dialysis treatment or a kidney transplant), and escalating medical costs.

It was reported previously (F. Toegel et al. Kidney International 62:1772-1784, 2005) that experimental AKI causes a marked up regulation of the chemokine SDF-1 (CXCL12) in the mouse kidney, mediating the homing of cells that express CXCR4, the cognate receptor for SDF-1. Both hematopoietic stem cells (HSC), endothelial precursor cells (EPC) and mesenchymal stem cells or Multipotent Stromal Cells (MSC) express CXCR4, and their recruitment to the injured kidney can be blocked by a neutralizing antibody to CXCR4 or AMD3100, a specific blocker of CXCR4. Physiologically, SDF-1 levels are highest in bone marrow niches, thereby facilitating the recruitment and engraftment of a bone marrow transplant. In experimental AKI, the renal levels of SDF-1 exceed those in the bone marrow, which facilitates the recruitment of MSC that are given for the prevention and treatment of AKI.

Other novel biomarkers for the early diagnosis of AKI have already been tested in humans including NGAL, IL-18, KIM-1, and L-type Fatty Acid Binding Protein (J M Thurman et al. Kidney International 73:379-81 (2008); C R Parikh et al. Kidney International 73:801-3 (2008); and W H Han et al. Kidney International 73:863-9 (2008)). These biomarkers have also been shown to possess diagnostic and some prognostic utility in experimental and clinical Acute Renal Failure. However, distinct from the above biomarkers, the robust upregulation of SDF-1 in the kidney with AKI, and its early release (within 2 hrs post injury) into the urine, specifically diagnoses AKI, and importantly, and simultaneously identifies the time point when MSC-based therapy is most effective and indicated.

### SUMMARY OF THE INVENTION

According to some embodiments, the invention provides methods of detecting a likelihood of or diagnosing acute kidney injury (AKI) in a patient by providing a normal level, amount or concentration of SDF-1 in urine of the patient, measuring the amount, level or concentration of SDF-1 in a urine sample procured from the patient, wherein if the SDF-1 amount, level or concentration is higher in the urine sample than the normal level, amount or concentration of SDF-1, the patient is likely to have AKI. According to some embodiments, the normal level, amount or concentration of SDF-1 in urine of the patient is determined by measuring the amount, level or concentration of SDF-1 in the patient at a time prior to the patient suffering from AKI. In some embodiments, the normal amount, level or concentration of SDF-1 is measured in urine from several subjects to determine the normal amount, level or concentration of SDF-1. In some embodiments, enzyme linked immunosorbent assay (ELISA) is used to detect the amount, level or concentration of SDF-1. In some embodiments, the urinary SDF-1 is normalized for urinary creatinine.

The invention also provides a method of detecting a likelihood of or diagnosing multiorgan failure (MOF) in a patient by providing a normal level, amount or concentration of SDF-1 in urine of the patient, providing a normal level, amount or concentration of SDF-1 in serum of the patient, measuring the amount, level or concentration of SDF-1 in a urine sample and a serum sample procured from the patient, wherein if the SDF-1 amount, level or concentration is higher in the urine sample and the serum sample than the normal level, amount or concentration of SDF-1 in urine or serum, the patient is likely to have MOF. According to some embodiments, the normal level, amount or concentration of SDF-1 in urine or serum of the patient is determined by measuring the amount, level or concentration of SDF-1 in the patient at a time prior to the patient suffering from MOF. In some embodiments, the normal amount, level or concentration of SDF-1 is measured in urine or serum from several subjects to determine the normal amount, level or concentration of SDF-1. In some embodiments, enzyme linked immunosorbent assay (ELISA) is used to detect the amount, level or concentration of SDF-1. In some embodiments, the urinary SDF-1 is normalized for urinary creatinine.

The invention also provides a method of treating AKI in a patient by providing a normal level, amount or concentration of SDF-1 in urine of the patient, measuring the amount, level or concentration of SDF-1 in a urine sample procured from the patient, wherein if the SDF-1 amount, level or concentration is higher in the urine sample and the serum sample than the normal level, a therapeutically effective amount of mesenchymal stem cells (MSC) are administered to the patient, thereby treating the AKI in the patient. According to some embodiments, the normal level, amount or concentration of SDF-1 in urine or serum of the patient is determined by measuring the amount, level or concentration of SDF-1 in the patient at a time prior to the patient suffering from AKI. In some embodiments, the normal amount, level or concentration of SDF-1 is measured in urine or serum from several subjects to determine the normal amount, level or concentration of SDF-1. In some embodiments, enzyme linked immunosorbent assay (ELISA) is used to detect the amount, level or concentration of SDF-1. In some embodiments, the urinary SDF-1 is normalized for urinary creatinine. In some embodiments, the therapeutic dose of MSC is between about 1×10⁵ and 1.5×10⁶ cells. In some embodiments, the MSC are isolated from a density gradient, wherein the density of the gradient from which the MSC is between 1.050 and 1.070 g/ml. In some embodiments, the MSC are cultured in platelet lysate (PL) prior to administration.

The invention also provides a method of treating AKI in a patient by providing a normal level, amount or concentration of SDF-1 in urine of the patient, administering a CD26 measuring the amount, level or concentration of SDF-1 in a urine sample procured from the patient, wherein if the SDF-1 amount, level or concentration is higher in the urine sample and the serum sample than the normal level, a therapeutically effective amount of mesenchymal stem cells (MSC) is administered to the patient, thereby treating the AKI in the patient. According to some embodiments, the normal level, amount or concentration of SDF-1 in urine or serum of the patient is determined by measuring the amount, level or concentration of SDF-1 in the patient at a time prior to the patient suffering from AKI. In some embodiments, the normal amount, level or concentration of SDF-1 is measured in urine or serum from several subjects to determine the normal amount, level or concentration of SDF-1. In some embodiments, enzyme linked immunosorbent assay (ELISA) is used to detect the amount, level or concentration of SDF-1. In some embodiments, the urinary SDF-1 is normalized for urinary creatinine. In some embodiments, the therapeutic dose of MSC is between about 1×10⁵ and 1.5×10⁶ cells. In some embodiments, the MSC are isolated from a density gradient, wherein the density of the gradient from which the MSC is between 1.050 and 1.070 g/ml. In some embodiments, the MSC are cultured in platelet lysate (PL) prior to administration.

According to some embodiments, the invention also provides methods of transplanting a kidney from a donor to a patient by providing a normal level, amount or concentration of SDF-1 in urine of the donor, measuring the amount, level or concentration of SDF-1 in a urine sample procured from the donor, wherein if the SDF-1 amount, level or concentration is higher in the urine sample and the serum sample than the normal level, a therapeutically effective amount of mesenchymal stem cells (MSC) are administered to the patient when the kidney is transplanted. According to some embodiments, the normal level, amount or concentration of SDF-1 in urine or serum of the patient is determined by measuring the amount, level or concentration of SDF-1 in the patient at a time prior to the patient suffering from AKI. In some embodiments, the normal amount, level or concentration of SDF-1 is measured in urine or serum from several subjects to determine the normal amount, level or concentration of SDF-1. In some embodiments, enzyme linked immunosorbent assay (ELISA) is used to detect the amount, level or concentration of SDF-1. In some embodiments, the urinary SDF-1 is normalized for urinary creatinine. In some embodiments, the therapeutic dose of MSC is between about 1×10⁵ and 1.5×10⁶ cells. In some embodiments, the MSC are isolated from a density gradient, wherein the density of the gradient from which the MSC is between 1.050 and 1.070 g/ml. In some embodiments, the MSC are cultured in platelet lysate (PL) prior to administration.

### BRIEF DESCRIPTION OF FIGURES

FIG. 1 is a bar graph showing the concentration of serum creatinine in rats at various time points before and after acute kidney injury (AKI).

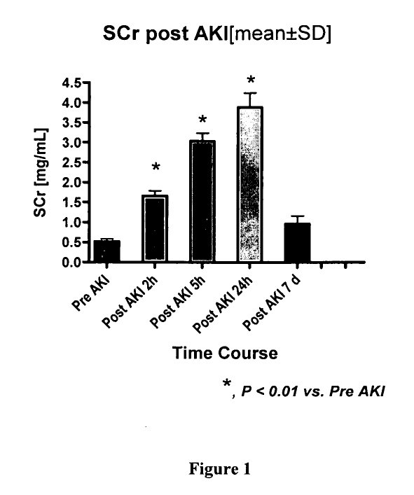

FIG. 2 is a bar graph showing the concentration of plasma SDF-1 in rats at various time points before and after AKI.

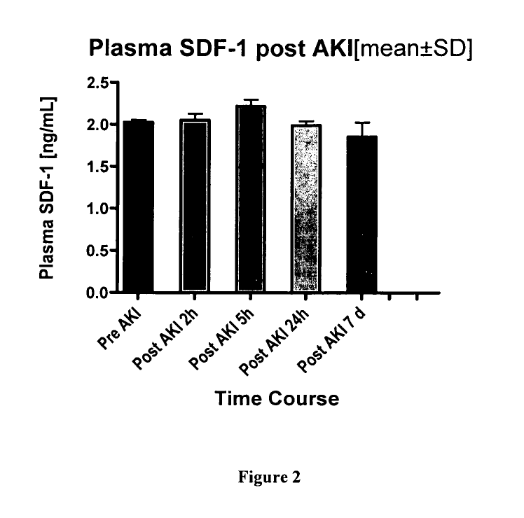

FIG. 3 is a bar graph showing the concentration of urine SDF-1 in rats at various time points before and after AKI.

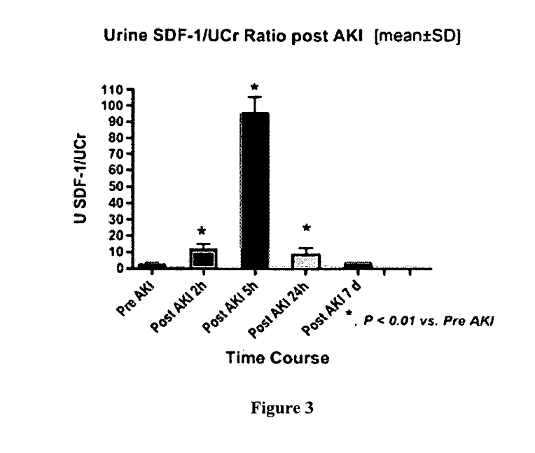

### DETAILED DESCRIPTION OF THE PREFERRED EMBODIMENTS

Before the present proteins, nucleotide sequences, peptides, etc., and methods are described, it is understood that this invention is not limited to the particular methodology, protocols, and reagents described, as these may vary. It also is to be understood that the terminology used herein is for the purpose of describing particular embodiments only, and is not intended to limit the scope of the present invention which will be limited only by the appended claims.

Accordingly, the current invention demonstrates that a documented rise in urinary SDF-1 levels provides a new tool to diagnose Acute Kidney Injury (AKI) within two hours after a renal insult. The methods provided herein allow the early institution of renoprotective therapy, and uniquely identifies the time point after renal injury at which the administration of MSC is most effective to treat kidney injury. It has been shown in International Application No. PCT/US08/001,371, incorporated herein by reference in its entirety, that specific drugs are useful to prolong the renal expression of SDF-1 by blocking its CD26 (DipeptidylPeptidase IV)-mediated inactivation in the kidney, thereby potentiating the homing and kidney protective activity of administered MSC in AKI. Importantly, such intervention is also expected to have an MSC sparing effect, i.e., allowing the same beneficial effects to be achieved with lower numbers of MSC. Thus, SDF-1 is important for the effectiveness of MSC therapy and the detection of high levels of SDF-1 in urine indicates a favorable time for the administration of MSC.

A rise of urinary SDF-1 levels also occurs at the time of kidney harvest from a cadaveric donor. An elevated SDF-1 level in a kidney from a living donor can both be used to diagnose AKI of the donor kidney and identify the utility of MSC therapy at the time of organ implantation, thereby ameliorating post-operative AKI (delayed graft function) and increased graft loss due to AKI-induced rise in subsequent graft loss due to rejection.

The simultaneous determination of SDF-1 levels in both urine and blood provides a highly useful tool that allows for the distinction between acute renal injury (increased urine levels without a major rise in blood levels) and injury of extrarenal organs such as liver, brain, heart, lungs, pancreas and others (rise in blood levels without a renal contribution). In the setting of multiorgan failure (MOP), both blood and urine levels of SDF-1 are elevated, indicating injury of multiple organs. MOF develops in the most severely ill patients who have sepsis, particularly when the latter develops after major surgery or trauma. It occurs also with greater frequency and severity in elderly patients, those with diabetes mellitus, underlying cardiovascular disease and impaired immune defenses. MOF is characterized by shock, AKI, leaky cell membranes, dysfunction of lungs, liver, heart, blood vessels and other organs. Mortality due to MOF approaches 100% despite the utilization of the most aggressive forms of therapy, including intubation and ventilatory support, administration of vasopressors and antibiotics, steroids, hemodialysis and parenteral nutrition. Many of these patients have serious impairment of the healing of surgical or trauma wound, and, when infected, these wounds further contribute to recurrent infections, morbidity and death.

SDF-1 Profiling (urine and blood) has the following advantages over the current state of art. It allows for the very early diagnosis of AKI. The peak rise in urinary SDF-1 after AKI indicates when AKI therapy is most effective. Any kidney therapy can be directed by SDF-1 profiling. One example of AKI therapy is MSC therapy. The peak rise in urinary SDF-1 after AKI indicates when MSC therapy is most effective. Examples of alternative therapies that are used with the SDF-1 profiling methods of the invention include those described in International Publication Nos. WO 04/044142 and WO 08/042,174, incorporated herein by reference in their entireties. SDF-1 profiling allows for the distinction between AKI and the injury of other major organs (heart, brain, liver, lungs, pancreas and others). It allows for the early assessment of renal injury in a cadaveric kidney donor, and simultaneously identifies the efficacy of MSC administration post implantation. Elevated urinary SDF-1 levels prior to a high risk procedure (cardiac surgery) predict poor renal outcome and indicate that MSC therapy is effective and needed. Elevated urinary SDF-1 may indicate that a patient with chronic kidney diseases (diabetes mellitus, glomerulonephritis, hypertension, etc.) has progressive disease and may respond to MSC therapy.

SDF-1 profiling provides a straight forward diagnostic, prognostic and therapy-specific test in patients suspected of having or for being at risk for AKI, allowing for early and specific institution of treatment (MSC therapy). Such information is of great utility in a very large number of patients world wide.

SDF-1 profiling also provides distinction between kidney injury and injury of other major organs, because of the characteristic changes in urine and blood levels. This is of particular utility in intensive care unit patients.

SDF-1 profiling allows for the assessment of the health of a transplant kidney obtained from a cadaveric donor, and indicates when MSC therapy post implantation will improve outcome.

SDF-1 profiling post MSC therapy can be used to determine whether additional MSC therapy is needed in patients with AKI, post kidney transplant and in patients with progressive chronic kidney disease or injury of other major organs.

In summary, SDF-1 profiling provides a completely unique biomarker of high diagnostic, prognostic and therapeutic value in a very large number of patients world wide. Its ability to diagnose AKI early together with identifying the time point when specific therapy (MSC administration) is most effective, fundamentally distinguishes this biomarker from others (e.g., NGAL, IL-18, KIM-1, L-type fatty acid binding protein). The latter identify kidney injury early, but do not provide information about the specific type of intervention that will be most effective at a given time point.

Determining SDF-1 Amounts

The amount of SDF-1 in the urine, serum or any other bodily fluid of a patient may be measured using any assay known in the art used to detect protein concentration and/or the presence the absence of specific proteins. Methods of SDF-1 protein detection include, but are not limited to, Western blot immunoassay, immunohistology, fluorescence activated cell sorting (FACS), radioimmunoassay (RIA), fluorescent immunoassay, enzyme linked immunosorbent assay (ELISA), or an immunoassay that uses a solid support, e.g., latex beads.

According to some embodiments, control samples from patients without kidney or organ pathology are assigned a relative SDF-1 amount or concentration value of 1. In preferred embodiments, urinary SDF-1 amounts, levels and concentrations are normalized to urinary creatinine amounts, levels and concentrations. In this case, the amount of increase in SDF-1 in patients or subjects suffering from or subjected to AKI can increase at least 2 fold, 5 fold, 10 fold, 20 fold, 30 fold, 40 fold, 50 fold, 60 fold, 70 fold, 80 fold, 90 fold and 100 fold. The fold increase is dependent upon the amount of time between in start of AKI and when a sample of urine is taken from the patient or subject suffering from or subject to AKI. The highest fold induction of SDF-1 over control should be between 2 and 24 hours post AKI. The SDF-1 amount, level or concentration in the urine of the patient or subject should gradually decline after this time period.

The effects of the compounds upon kidney or organ pathology can be measured by detecting the amount of concentration of SDF-1 in the bodily fluids of patients, preferably serum or urine. Any suitable physiological change that affects SDF-1 amount or concentration in a bodily fluid of a patient can be detected according to the methods of the invention. Preferably, the kidney pathology is acute kidney injury (AKI).

Moreover, the appropriate timing for the administration of MSC for the treatment of kidney or other organ pathology can be measured by detecting the amount of concentration of SDF-1 in the bodily fluids of patients, preferably serum or urine. It has been shown that ischemia-reperfusion injury (IRI) causes the renal levels of SDF-1 (CXCL12) to rapidly rise above those in the bone marrow. This potentiates the renal homing of CXCR4-expressing (SDF-1 receptor) cells, such as administered mesenchymal stem cells (MSC), circulating Endothelial Precursor Cells, and others. MSCs, and their administration immediately or 24 hrs after AKI, robustly protects renal function and hastens renal repair through complex paracrine mechanisms. Accordingly, a significant rise in the urinary SDF-1/creatinine concentration ratio post AKI facilitates the early diagnosis of AKI and simultaneously indicates that homing of MSC to the kidney, if given at this time point, will be potentiated. This, in turn, results in optimized kidney protection and repair.

Medical Use

The compositions of this invention are useful for detecting and/or diagnosing organ or kidney pathology through the detection of SDF-1 in bodily fluids of a patient. Preferably, these body fluids are serum and/or urine. Kidney pathologies include acute kidney injury (AKI). AKI can be caused by pre-renal causes including decreased blood volume, hepatorenal syndrome, vascular pathologies, and infection. AKI can also be caused by renal causes including toxins, rhabdomyolysis, hemolysis, multiple myeloma and acute glomerulonephritis. AKI can also be caused by post renal causes including medication that interferes with the normal bladder emptying, prostate cancer, kidney stones, abdominal malignancy or an obstructed urinary catheter. Various injuries of other major organs in the context of multiorgan failure, often initiated by AKI, or per se, also causes up-regulation of SDF-1.

The compositions of the invention are also useful for timing the administration of MSC therapy in connection with AKI. When SDF-1 levels are high in the urine of a patient, MSC should be administered for the treatment of AKI. Moreover, a CD26 inhibitor can be administered to the patient and the SDF-1 levels in the urine of the patient determined in the patient, wherein when the SDF-1 levels are high, MSC therapy is administered to the patient. The compositions of the invention are also useful for determining whether a kidney to be transplanted should be transplanted with a dose of MSC. If donor urine contains a high amount of SDF-1, then when the donor's kidney is transplanted to the patient, it is preferably co-adminstered with a therapeutically effective dose of MSC.

In addition, since pharmacological inhibition of CD26 (dipeptidyl peptidase IV), the principal enzyme that inactivates SDF-1 in the kidney and elsewhere, is readily possible with drugs that are in clinical use (e.g., sitagliptin, Januvia™), treatment of a patient with Aki both with a CD26 inhibitor and MSC, will augment SDF-1-mediated recruitment of administered MSC to the kidney, which, in turn, potentiates their renoprotective efficacy while requiring lower numbers of MSC. This combination therapy will thus be advantageous for the patient and also reduce the production costs of MSC.

The function of a donor kidney at the time of harvest and at the time of implantation in a recipient determines the subsequent degree of delayed graft function (DGF), i.e., the severity of post-transplant AKI. This, in turn, determines both the early outcome in a kidney transplant recipient (need for dialysis, increased length of hospital stay, morbidity and mortality of AKI) and subsequent frequency of graft loss due to rejection. Measurement of urinary SDF-1 and creatinine levels at time of kidney harvest, at time of implantation, and following transplantation will allow a prognostic assessment of graft function in the recipient, and determine that MSC administration per se, or together with a CD26 inhibitor, will be beneficial in protecting against significant DGF and the secondary rise of subsequent graft loss due to rejection.

Mesenchymal Stem Cells (MSC)

MSC according to the invention are described, for example, in U.S. Publication No. 20070178071, incorporated herein by reference in its entirety. The culturing of MSC in platelet lysate (PL) is described in greater detail in U.S. Provisional Patent Application No. 61/086,033, also incorporated herein by reference in its entirety. Certain embodiments of therapeutically effective dosages of MSC are described in greater detail in U.S. patent application Ser. No. 11/913,900, also incorporated herein by reference in its entirety. The use of a CD26 inhibitor in order to potentiate the therapeutic effect of MSC is described in International Application No. PCT/US08/001,371, also incorporated herein by reference in its entirety. Certain embodiments of isolation of MSC with density gradients are shown in U.S. Publication No. 20070160583, also incorporated herein by reference in its entirety.

Definitions

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only not intended to be limiting. Other features and advantages of the invention will be apparent from the following detailed description and claims.

For the purposes of promoting an understanding of the embodiments described herein, reference will be made to preferred embodiments and specific language will be used to describe the same. The terminology used herein is for the purpose of describing particular embodiments only, and is not intended to limit the scope of the present invention. As used throughout this disclosure, the singular forms “a,” “an,” and “the” include plural reference unless the context clearly dictates otherwise. Thus, for example, a reference to “a composition” includes a plurality of such compositions, as well as a single composition, and a reference to “a therapeutic agent” is a reference to one or more therapeutic and/or pharmaceutical agents and equivalents thereof known to those skilled in the art, and so forth. Thus, for example, a reference to “a host cell” includes a plurality of such host cells, and a reference to “an antibody” is a reference to one or more antibodies and equivalents thereof known to those skilled in the art, and so forth.

For the purposes of promoting an understanding of the embodiments described herein, reference will be made to what high or higher amounts of SDF-1 in patient or subject samples mean. This invention is based on the unexpected finding that while SDF-1 amount, level and/or concentration is not significantly elevated in the blood or serum of patients or subjects with AKI, SDF-1 amount, level and/or concentration in the urine of patients and subjects with AKI is significantly elevated. This invention is also based on the unexpected finding that SDF-1 amount, level and/or concentration is significantly elevated in the blood or serum and urine of patients or subjects with multiorgan failure (MOF).

In some embodiments, the amount of SDF-1 is normalized to urinary creatinine amounts, levels and/or concentrations. In this case, the amount of increase in SDF-1 in patients or subjects suffering from or subjected to AKI can increase at least 2 fold, at least 5 fold, at least 10 fold, at least 20 fold, at least 30 fold, at least 40 fold, at least 50 fold, at least 60 fold, at least 70 fold, at least 80 fold, at least 90 fold and at least 100 fold. The fold increase is dependent upon the amount of time between the start of AKI and when a sample of urine is taken from the patient or subject suffering from or subject to AKI. The highest fold induction of SDF-1 over control should be between 2 and 24 hours post AKI (e.g., 4, 6, 8, 10, 12, 14, 16, 18, 20, and/or 22 hours post AKI). The SDF-1 amount, level or concentration in the urine of the patient or subject should gradually decline after this time period.

### EXAMPLES

The following examples are illustrative, but not limiting, of the methods and compositions of the present invention. Other suitable modifications and adaptations of the variety of conditions and parameters normally encountered in therapy and that are obvious to those skilled in the art are within the spirit and scope of the embodiments.

### Example 1

### SDF-1 Levels are Increased in Rats Subject to Acute Renal Injury

Using a standard ischemia/reperfusion model of AKI in rats, e.g. temporary clamping of both renal arteries, followed by reperfusion after clamp removal, serum and urinary SDF-1 levels (specific ELISA; R&D Systems), together with serum and urinary creatinine levels, were monitored at 2, 5, 12, 24, 48 and 72 hrs after induction of AKI, and again at 7 days following AKI. Serum levels of SDF-1 rose only minimally at these time points, while SCr levels rose progressively over 72 hrs and gradually fell towards baseline at 7 days. In contrast, urinary SDF-1 levels, normalized for urinary creatinine, rose highly significantly at 2, 5, 12 and 24 hrs, gradually declining thereafter. Urinary SDF-1/creatinine concentration ratios significantly increased by 13-fold at 2 hrs, 68-fold by 5 hrs, 4-fold by 24 hrs, and 1.7-fold on day 7 post IRI (vs. baseline). This demonstrates that renally produced SDF-1 is released into the urine. Since blood levels of SDF-1 remain essentially unchanged as renal function deteriorates, the contribution of blood SDF-1 to urinary SDF-1 levels is negligible. Current studies further define the SDF-1 expression profiles after AKI in rats in serum, kidney (mRNA, protein), and urine.

### Example 2

### MSC Administered to Rats Subject to Acute Renal Injury when SDF-1 Levels are Increased Urine Samples Results in Protected Renal Function and Accelerated Recovery

Using the same AKI model in rats, MSC were administered as before (F. Toegel C. Westenfelder. Am J Physiol Renal Physiol 289:F31-F42, 2005) when urinary SDF-1 levels were at their peak (˜5 hrs post AKI) and when SDF-1 levels began to fall (after 24 hrs). The early administration of MSC was most effective in protecting renal function and in accelerating recovery of renal function.

### Example 3

### SDF-1 Levels are Increased in Humans Who Suffer from Acute Renal Injury

Serum and urinary SDF-1 levels together with serum and urinary creatinine levels, will be monitored in human subject who suffer from acute kidney injury (AKI), and again at 7 days following AKI and compared to SDF-1 levels in human subjects who do not experience AKI. Serum levels of SDF-1 are expected to rise only minimally at these time points, while SCr levels will rise progressively over 72 hrs and gradually fell towards baseline at 7 days. In contrast, we expect urinary SDF-1 levels, normalized for urinary creatinine, to rise significantly at 2, 5, 12 and 24 hrs, gradually declining thereafter.

### Example 4

### Blood, Kidney and Urinary SDF-1 Expression Levels in AKI in Rats Following MSC Administration

Blood, urinary and kidney SDF-1 levels will be monitored in rats subjected to AKI, e.g. temporary clamping of both renal arteries, followed by reperfusion after clamp removal. SDF-1 levels will be monitored at 2, 5, 12, 24, 48 and 72 hrs after induction of AKI, and again at 7 days following AKI. We expect that serum levels of SDF-1 will rise only minimally at these time points, while kidney and urine levels of SDF-1 will significantly at 2, 5, 12 and 24 hrs, gradually declining thereafter.

### Example 5

### Blood, Kidney and Urinary SDF-1 Expression Levels in a Chronic Kidney Disease Model in Rats Following MSC Administration

Blood, urinary and kidney SDF-1 levels will be monitored in rats subjected to a chronic kidney disease model. SDF-1 levels will be monitored at various times after induction of chronic kidney disease, and again at 7 days following induction of chronic kidney disease. We will also assess the expression of SDF-1 in blood, urine and kidney in rats subject to chronic kidney disease and treated with MSC therapy.

### Example 6

### Blood, Kidney and Urinary SDF-1 Expression Levels in Rats that Serve as Donors for a Subsequent Kidney Transplant with and without MSC Administration

Blood, urinary and kidney SDF-1 levels will be monitored in rats that will serve as kidney donors. We will also assess the expression of SDF-1 in blood, urine and kidney in rats that will serve as kidney donors that are treated with MSC therapy.

### Example 7

### Blood, Kidney and Urinary SDF-1 Expression Levels in Humans that Serve as Donors for a Subsequent Kidney Transplant with and without MSC Administration

Blood, urinary and kidney SDF-1 levels will be monitored in humans that serve as kidney donors. We will also assess the expression of SDF-1 in blood, urine and kidney in human subjects that serve as kidney donors that are treated with MSC therapy.

1. A method of detecting a likelihood of a disease in a patient in need thereof comprising:
(a) providing a normal level, amount or concentration of SDF-1 in urine of the patient; and
(b) measuring the amount, level or concentration of SDF-1 in a urine sample procured from the patient,
wherein if the SDF-1 amount, level or concentration is higher in the urine sample than the normal level, amount or concentration of SDF-1, the patient is likely to have acute kidney injury (AKI).

2. The method of claim 1, wherein the normal level, amount or concentration of SDF-1 in urine of the patient is determined by measuring the amount, level or concentration of SDF-1 in the patient at a time prior to the patient suffering from AKI.

3. The method of claim 1, wherein the normal amount, level or concentration of SDF-1 is measured in urine from several subjects to determine the normal amount, level or concentration of SDF-1.

4. (canceled)

5. The method of claim 1, wherein the urinary SDF-1 is normalized for urinary creatinine.

6. The method of claim 1 further comprising:
(c) providing a normal level, amount or concentration of SDF-1 in serum of the patient; and
(d) measuring the amount, level or concentration of SDF-1 in a serum sample procured from the patient;
wherein if the SDF-1 amount, level or concentration is higher in the urine sample and the serum sample than the normal level, amount or concentration of SDF-1 in urine or serum, the patient is likely to have multi-organ failure (MOF).

7-10. (canceled)

11. A method of treating AKI in a patient in need thereof comprising:
(a) providing a normal level, amount or concentration of SDF-1 in urine of the patient; and
(b) measuring the amount, level or concentration of SDF-1 in a urine sample procured from the patient,
wherein if the SDF-1 amount, level or concentration is higher in the urine sample and the serum sample than the normal level, a therapeutically effective amount of mesenchymal stem cells (MSC) are administered to the patient, thereby treating the AKI in the patient.

12. The method of claim 11, wherein the normal level, amount or concentration of SDF-1 in urine or serum of the patient is determined by measuring the amount, level or concentration of SDF-1 in the patient at a time prior to the patient suffering from AKI.

13. The method of claim 11, wherein the normal amount, level or concentration of SDF-1 is measured in urine or serum from several subjects to determine the normal amount, level or concentration of SDF-1.

14. (canceled)

15. The method of claim 11, wherein the urinary SDF-1 is normalized for urinary creatinine.

16. The method of claim 11, wherein the therapeutic dose of MSC is between about 1×10⁵ and 1.5×10⁶ cells.

17. The method of claim 11, wherein the MSC are isolated from a density gradient, wherein the density of the gradient from which the MSC is between 1.050 and 1.070 g/ml.

18. The method of claim 11, wherein the MSC are cultured in platelet lysate (PL) prior to administration.

19. The method of claim 11 further comprising:
administering a CD26 inhibitor to the patient.

20-26. (canceled)

27. A method of transplanting a kidney from a donor to a patient comprising:
(a) providing a normal level, amount or concentration of SDF-1 in urine of the donor; and
(b) measuring the amount, level or concentration of SDF-1 in a urine sample procured from the donor,
wherein if the SDF-1 amount, level or concentration is higher in the urine sample and the serum sample than the normal level, a therapeutically effective amount of mesenchymal stem cells (MSC) are administered to the patient when the kidney is transplanted.

28. The method of claim 27, wherein the normal level, amount or concentration of SDF-1 in urine or serum of the patient is determined by measuring the amount, level or concentration of SDF-1 in the patient at a time prior to the patient suffering from AKI.

29. The method of claim 27, wherein the normal amount, level or concentration of SDF-1 is measured in urine or serum from several subjects to determine the normal amount, level or concentration of SDF-1.

30. (canceled)

31. The method of claim 27, wherein the urinary SDF-1 is normalized for urinary creatinine.

32. The method of claim 27, wherein the therapeutic dose of MSC is between about 1×10⁵ and 1.5×10⁶ cells.

33. The method of claim 27, wherein the MSC are isolated from a density gradient, wherein the density of the gradient from which the MSC is between 1.050 and 1.070 g/ml.

34. The method of claim 27, wherein the MSC are cultured in platelet lysate (PL) prior to administration.
